(19) United States
(12) Patent Application Publication
(10) Pub. No.: US 2023/0025893 A1
(43) Pub. Date: Jan. 26, 2023

(54) HOT MELT ADHESIVE COMPOSITIONS INCLUDING AN ENVIRONMENTALLY CONSCIOUS ALIPHATIC PLASTICIZER

(71) Applicant: H.B. Fuller Company, St. Paul, MN (US)

(72) Inventors: Haiying Zhou, Arden Hills, MN (US); Andrew R. Michel, Plymouth, MN (US)

(21) Appl. No.: 17/812,631

(22) Filed: Jul. 14, 2022

Related U.S. Application Data
(60) Provisional application No. 63/203,250, filed on Jul. 14, 2021.
(60) Provisional application No. 63/203,253, filed on Jul. 14, 2021.

(51) Int. Cl. C09J 7/35; C09J 11/08; C09J 7/21; C09J 123/02; C09J 11/06; C09J 125/08

(52) U.S. Cl. CPC C09J 7/35; C09J 11/08; C09J 7/21; C09J 123/025; C09J 11/06; C09J 125/08; C09J 2301/122; C09J 2400/263; C09J 2301/304

(57) ABSTRACT

The inventors have discovered hot melt adhesive compositions that can be formulated to have a high percentage of environmentally conscious components and still provide performance over a broad temperature range.

The invention features a hot melt adhesive composition including from 5% by weight to 50% by weight of a thermoplastic polymer, from 10% by weight to 80% by weight of a bio-based tackifying agent, and from 2% by weight to 50% by weight of an environmentally conscious aliphatic plasticizer having a cyclo-aliphatic content of no more than 2% by weight as tested by ¹H-NMR Spectroscopy.

### CROSS REFERENCE TO RELATED APPLICATIONS

This application claims the benefit of U.S. Provisional Application No. 63/203,250, filed Jul. 14, 2021 and U.S. Provisional Application No. 63/203,253, filed Jul. 14, 2021 which is incorporated herein.

### BACKGROUND

In the area of industrial adhesives, hot melt adhesive compositions are commonly used to bond together a wide variety of articles including tapes, labels, cases, cartons and disposable absorbent articles comprising non-woven substrates e.g. adult incontinence products, disposable diapers, sanitary napkins, bed pads, puppy pads, medical dressings, etc.

Hot melt adhesive compositions include materials such as polymers, tackifying agents, plasticizers and waxes. Such materials are commonly derived from petroleum based feedstocks. In recent years, there has been a demand for hot melt adhesive compositions derived from bio-based materials such as rosin based tackifying agents and terpene based tackifying agents. There is a need for a hot melt including bio-based tackifying agents that has improved functionality over a broad temperature range.

### SUMMARY

In one aspect, the invention features a hot melt adhesive composition including from 5% by weight to 50% by weight of a thermoplastic polymer, from 10% by weight to 80% by weight of a bio-based tackifying agent, and from 2% by weight to 50% by weight of an environmentally conscious aliphatic plasticizer having a cyclo-aliphatic content of no more than 2% by weight as tested by ¹H-NMR Spectroscopy.

In one embodiment, the thermoplastic polymer is selected from the group consisting of olefin polymer, styrene block copolymer, functionalized versions thereof and combinations thereof. In another embodiment, the hot melt adhesive composition has a bio-based component content of at least 50% by weight, at least 70% by weight, or even from 65% by weight to 100% by weight.

In one embodiment, the environmentally conscious aliphatic plasticizer is a bio-based aliphatic plasticizer is selected from the group consisted of linear alkanes, branched alkanes, or combinations thereof. In another embodiment, the bio-based aliphatic plasticizer is derived from plant oil. In a different embodiment, the bio-based aliphatic plasticizer is a hydrogenated reaction product of octadecane and hexadecane. In one embodiment, the bio-based aliphatic plasticizer is produced or derived from renewable resources. In a different embodiment, the bio-based aliphatic plasticizer has a cyclo-aliphatic content of no more than 1% by weight as tested by ¹H-NMR Spectroscopy, or even no more than 0.5% by weight as tested by ¹H-NMR Spectroscopy.

In one embodiment, the bio-based tackifying agent has a Ring and Ball softening point as reported by the supplier of from 80° C. to 120° C. In another embodiment, the bio-based tackifying agent has a neat Molten Gardner Color of from 0 to 4. In another embodiment, the bio-based tackifying agent is selected from the group consisting of rosin-based tackifying agent and terpene-based tackifying agent. In a different embodiment, the bio-based tackifying agent is a terpene-based tackifying agent. In one embodiment, the bio-based tackifying agent is a rosin-based tackifying agent. In another embodiment, the rosin based tackifying agent has a neat Molten Gardner Color of from 0 to 2. In another embodiment, 70% by weight to 100% by weight of the bio-based tackifying agent is produced or derived from renewable resources.

In one embodiment, the thermoplastic polymer is selected from the group consisting of environmentally conscious, bio-based and thermoplastic polymers considered sustainable by the mass balance approach. In another embodiment, the thermoplastic polymer is a styrene block copolymer. In one embodiment, the styrene block copolymer is selected from the group consisting of styrene-butadiene-styrene, styrene-isoprene-styrene, and combinations thereof. In another embodiment, the styrene block copolymer has an average styrene content of from 20% by weight to 70% by weight. In a different embodiment, the styrene block copolymer has an average styrene content of from 20% by weight to 45% by weight.

In one aspect, the hot melt adhesive composition includes from 10% by weight to 40% by weight of the thermoplastic polymer, the thermoplastic polymer comprising styrene block copolymer, from 15% by weight to 75% by weight of the bio-based tackifying agent, and from 5% by weight to 40% by weight of the environmentally conscious aliphatic plasticizer having a cyclo-aliphatic content of no more than 2% by weight as tested by ¹H-NMR Spectroscopy.

In one embodiment, the invention features an article selected from the group consisting of a tape, a label and a disposable absorbent article including the inventive hot melt adhesive compositions. In a different embodiment, the article is selected from the group consisting of a paper tape and a paper label. In another embodiment, the invention features a disposable absorbent article including a first substrate, a second substrate, and the inventive hot melt adhesive composition, wherein the hot melt adhesive composition is disposed on at least one of the first and second substrates. In one embodiment, at least one of the substrates is bio-based. In another embodiment, the bio-based substrate is cotton.

The inventors have discovered hot melt adhesive compositions that can be formulated to have a high percentage of environmentally conscious or even bio-based components and still provide performance over a broad temperature range.

### DETAILED DESCRIPTION OF THE INVENTION

### Definitions

“Renewable resource” is used herein to refer to a resource that is produced by a natural process at a rate comparable to its rate of consumption. The resource can be replenished naturally or by engineered agricultural techniques. Examples of renewable resources include but are not limited to plants (e.g., sugar cane, beets, corn, potatoes, citrus fruit (e.g. oranges), woody plants, cellulosic waste, etc.), animals, fish, bacteria, fungi, and forestry products (e.g. pine and spruce trees). These resources can be naturally occurring, hybrids, or genetically engineered organisms. Natural resources such as crude oil, coal and natural gas are not considered renewable as they are derived from materials that will run out or will not be replenished for thousands or even millions of years.

“Bio-based” is used herein to refer to a component of the hot melt adhesive composition that is at least partially produced or is partially derived from a renewable resource.

“Environmentally conscious” is used herein to refer to a component of the hot melt adhesive composition that has at least one property selected from the group consisting of bio-based and having a total cradle to gate CO₂ emission value of less than 1.5 kgCO₂ e/kg as evaluated by ISO-14040/14044 (The total emission value is a sum of the biogenic and non-biogenic carbon emissions).

### Hot Melt Adhesive Composition

The invention features a hot melt adhesive composition including from 5% by weight to 50% by weight of a thermoplastic polymer, from 10% by weight to 80% by weight of a bio-based tackifying agent, and from 2% by weight to 50% by weight of an environmentally conscious aliphatic plasticizer having a cyclo-aliphatic content of no more than 2% by weight as tested by 1H-NMR Spectroscopy.

In another embodiment, the invention features a hot melt adhesive composition including from 10% by weight to 40% by weight of a thermoplastic polymer comprising styrene block copolymer, from 15% by weight to 75% by weight of a bio-based tackifying agent, and from 5% by weight to 40% by weight of an environmentally conscious aliphatic plasticizer having a cyclo-aliphatic content of no more than 2% by weight as tested by 1H-NMR Spectroscopy.

The invention also features a hot melt adhesive composition including from 5% by weight to 50% by weight of a thermoplastic polymer, from 10% by weight to 80% by weight of a bio-based tackifying agent, and from 2% by weight to 50% by weight of a bio-based aliphatic plasticizer having a cyclo-aliphatic content of no more than 2% by weight as tested by ¹H-NMR Spectroscopy.

In another embodiment, the invention features a hot melt adhesive composition including from 10% by weight to 40% by weight of a thermoplastic polymer comprising styrene block copolymer, from 15% by weight to 75% by weight of a bio-based tackifying agent, and from 5% by weight to 40% by weight of a bio-based aliphatic plasticizer having a cyclo-aliphatic content of no more than 2% by weight as tested by ¹H-NMR Spectroscopy.

In another embodiment, the invention features a hot melt adhesive composition including from 10% by weight to 30% by weight of a thermoplastic polymer comprising styrene block copolymer, from 45% by weight to 75% by weight of a bio-based tackifying agent, and from 10% by weight to 30% by weight of a bio-based aliphatic plasticizer having a cyclo-aliphatic content of no more than 2% by weight as tested by 1H-NMR Spectroscopy.

The hot melt adhesive composition can be free of petroleum derived tackifying agents. The hot melt adhesive composition can be free of petroleum derived tackifying agents and petroleum derived plasticizers.

The hot melt adhesive compositions of this invention include a high weight percent of environmentally conscious components. The environmentally conscious components can be bio-based.

The bio-based components are produced or derived from renewable resources. The bio-based components can be produced or derived from at least 25% by weight, at least 50% by weight, at least 75% by weight, at least 85% by weight, at least 90% by weight, at least 95% by weight, from 60% by weight to 85% by weight, from 60% by weight to 95% by weight, from 60% by weight to 100% by weight, from 65% by weight to 100% by weight, from 75% by weight to 100% by weight, from 80% by weight to 100% by weight or even 100% by weight (i.e. entirely) from renewable resources.

The bio-based components can have bio-based carbon content according to ASTM 6866-20 of at least 25%, at least 30%, at least 50%, at least 60%, at least 70%, at least 80%, at least 90%, from 25% to 100%, from 50% to 100%, from 70% to 100%, from 90% to 100%, or even 100% based on the total carbon content.

The hot melt adhesive composition can include at least 45% by weight, at least 50% by weight, at least 55% by weight, at least 60% by weight, at least 65% by weight, at least 70% by weight, at least 75%, at least 80% by weight, from 50% by weight to 95% by weight, from 50% by weight to 100% by weight, from 55% by weight to 100% by weight, from 60% by weight to 100% by weight, from 65% by weight to 100% by weight, from 70% by weight to 100% by weight, from 75% by weight to 100% by weight, from 80% by weight to 100% by weight, or even 100% by weight of components selected from the group consisting of having a total CO₂ emission of less than 1.5 kgCO₂ and bio-based.

Environmentally conscious components can have a total CO₂ emission of less than 1.5 kgCO₂, less than 1.0 kg CO₂ e/kg, less than 0.5 kg CO₂ e/kg, or even less than 0 kgCO₂ e/kg.

The hot melt adhesive compositions of this invention can have a Brookfield Viscosity at 149° C. of less than 15,000 cP, less than 10,000 cP, from 500 cP to 20,000 cP, or even from 500 cP to 15,000 cP.

The hot melt adhesive compositions of this invention utilize an environmentally conscious aliphatic plasticizer having a cycloaliphatic content of no greater than 2%, as tested by ¹H-NMR Spectroscopy. The inventors have discovered that as compared to prior art environmentally conscious plasticizers (that are primarily polar), this unique environmentally conscious aliphatic plasticizer can increase the bio-based content of the composition while still providing performance over a broad temperature range as indicated as by an increased Temperature Plateau Range as compared to compositions including prior art bio-based plasticizers (e.g., Control 1 versus Ex 1).

The Temperature Plateau Range is a predictor of the temperature range through which the hot melt adhesive composition has consistent cohesive strength. Further, an upper Temperature Plateau limit of greater than 60° C. is helpful to prevent cold flow during storage and shipping.

### Thermoplastic Polymer

The hot melt composition includes a thermoplastic polymer. The thermoplastic polymer can comprise one or more thermoplastic polymers.

The thermoplastic polymer can be environmentally conscious, or even bio-based. Bio-produced monomers can be used to make the bio-based thermoplastic polymer. It is anticipated that thermoplastic polymers made with bio-produced monomers will have similar properties to those made with petroleum derived monomers. The bio-produced monomers can be selected from the group consisting of ethylene, propylene, isoprene, butadiene, styrene, etc. However, useful bio-produced monomers are not restricted to this group. Bio-produced monomers are commonly derived from cellulose, starch and sugar e.g. glucose.

Alternatively, the thermoplastic polymer can be considered sustainable even when the bio-based origin cannot be detected by the radiocarbon method (ASTM 6866-20) due to time or dilution effects, but rather sustainability is demonstrated by relevant mass-balance methods (e.g. International Sustainability & Carbon Certification (ISCC) PLUS mass balance approach).

Alternatively, the thermoplastic polymer can be derived from petroleum-based materials. Environmentally conscious, bio-based, polymers considered sustainable by the mass balance approach and petroleum-based thermoplastic polymers can be combined in the present invention in any ratio, depending on cost and availability. Recycled thermoplastic polymers can also be used, alone or in combination with bio-based, sustainable polymers by mass balance approach and/or petroleum-based thermoplastic polymers.

The thermoplastic polymer can be selected from the group consisting of olefin polymer, styrene block copolymer, functionalized versions thereof (e.g., hydroxyl modified or maleic anhydride modified), and combinations thereof.

The olefin polymer can be selected from the group consisting of ethylene-based polymers (e.g., ethylene homopolymers and ethylene copolymers), propylene-based polymers (e.g., propylene homopolymers and propylene copolymers), functionalized versions thereof (e.g., hydroxyl modified or maleic anhydride modified), and combinations thereof.

The thermoplastic polymer can be prepared using a variety of catalysts including, e.g., a single site catalyst (e.g., metallocene catalysts (e.g., metallocene catalyzed ethylene alpha-olefin copolymers), constrained geometry catalysts (e.g., homogeneous linear or substantially linear ethylene alpha-olefin interpolymers prepared from ethylene and an alpha-olefin comonomer using a constrained geometry catalyst and having a polydispersity index of no greater than 2.5 and possessing long chain branching)), multiple single site catalysts, Ziegler-Natta catalysts and combinations thereof.

The thermoplastic polymer can include functional groups (i.e. be functionalized) including, e.g., carboxylic acid groups, anhydride groups (e.g., maleic anhydride), and combinations thereof.

If the hot melt adhesive composition includes a functionalized thermoplastic polymer, it can also include a second thermoplastic polymer that is not functionalized. The hot melt adhesive composition can include from 3% by weight to 25% by weight, from 5% by weight to 25% by weight, or even from 5% by weight to 15% by weight of the functionalized thermoplastic polymer. The hot melt adhesive composition can further include from 3% by weight to 25% by weight, from 5% by weight to 25% by weight, or even from 5% by weight to 15% by weight of the second thermoplastic polymer that is not functionalized.

If petroleum-based polymer is used, the amount of thermoplastic polymer can be limited to maximize the bio-based material content.

The hot melt adhesive composition can include from 3% by weight to 60% by weight, from 5% by weight to 60% by weight, from 10% by weight to 50% by weight, from 10% by weight to 40% by weight, from 10% by weight to 30% by weight, from 3% by weight to 25% by weight, from 10% by weight to 25% by weight, or even from 12% by weight to 20% by weight of thermoplastic polymer.

Olefin Polymer

The thermoplastic polymer can be an olefin polymer. The olefin polymer can be selected from the group consisting of ethylene-based polymer and propylene-based polymer.

The polymer can be an ethylene-based polymer having a density of no greater than 0.90 grams per cubic centimeter (g/cm3), or even no greater than 0.88 g/cm3. The ethylene-based polymer can be an ethylene alpha-olefin copolymer. The alpha-olefin monomer has at least three carbon atoms, or even from three to 20 carbon atoms, suitable examples of which include propylene, isobutylene, butene, pentene, hexene, heptene, octene, nonene, decene, dodecene, 4-methyl-1-pentene, 3-methyl pentene-1,3,5,5-trimethyl-hexene-1,5-ethyl-1-nonene, and combinations thereof. Specific examples of suitable ethylene copolymers include ethylene-propylene, ethylene-butene, ethylene-hexene, ethyene-octene, and combinations thereof.

Useful ethylene alpha-olefin copolymers are commercially available under of a variety of trade designations including, e.g., the AFFINITY series of trade designations from DowDuPont Chemical Company (Midland, Mich.) including, e.g., AFFINITY GA 1875, AFFINITY GA 1900, and AFFINITY GA 1950 ethylene-octene elastomers, AFFINITY GA 1000R maleic anhydride-modified ethylene-octene copolymer (which is also referred to as an interpolymer by the manufacturer), AFFINITY ethylene-propylene copolymers, and the ENGAGE series of trade designations from DowDuPont Chemical Company (Midland, Mich.) including ENGAGE 8200, ENGAGE 8401, and ENGAGE 8402 ethylene-octene copolymers.

The thermoplastic polymer can be a propylene-based polymer. The propylene-based polymer can be selected from the group consisting of a propylene alpha olefin copolymer and a propylene homopolymer.

The propylene-alpha-olefin copolymer is derived from propylene and at least one alpha-olefin co-monomer other than propylene (e.g., C2, and C4-C20 alpha-olefin co-monomers, and combinations thereof). Useful alpha-olefin co-monomers include, e.g., alpha-olefin monomers having at least two carbon atoms, at least four carbon atoms, from four carbon atoms to eight carbon atoms, and combinations thereof. Examples of suitable classes of alpha-olefin co-monomers include mono-alpha olefins (i.e., one unsaturated double bond) and higher order alpha olefins (e.g., dienes (e.g., 1,9-decadiene)). Suitable alpha-olefin monomers include, e.g., ethylene, butene, pentene, hexene, heptene, octene, nonene, decene, dodecene, 4-methyl-pentene-1,3-methyl pentene-1,3,5,5-trimethyl-hexene-1,5-ethyl-1-nonene, and combinations thereof. Specific examples of suitable propylene-alpha-olefin copolymers include propylene-ethylene, propylene-butene, propylene-hexene, propylene-octene, and combinations thereof.

Useful propylene-alpha-olefin copolymers include, e.g., copolymers, terpolymer, and higher order polymers, mixtures of at least two different propylene-alpha-olefin copolymers, and combinations thereof. Useful propylene-alpha-olefin co polymers also include, e.g., modified, unmodified, grafted, and ungrafted propylene-alpha-olefin copolymers, uni-modal propylene-alpha-olefin polymers, multi-modal propylene-alpha-olefin copolymers, and combinations thereof. The term “multi-modal” means the polymer has a multi-modal molecular weight distribution (weight average molecular weight (Mw)/number average molecular weight (Mn)) as determined by Size Exclusion Chromatography (SEC). Suitable commercially propylene-alpha-olefin copolymers are available under a variety of trade designations including, e.g., the VISTAMAXX series of trade designations from ExxonMobil Chemical Company (Houston, Tex.) including VISTAMAXX 6202 propylene-ethylene copolymer, VISTAMAXX 8880 propylene-ethylene copolymer, and VISTAMAXX 8380 propylene-ethylene copolymer. Suitable propylene homopolymers are commercially available under a variety of trade designations including, e.g., L-MODU S400 S410, S600 and S901, propylene homopolymers from Idemitsu Kosan Co., Ltd. (Japan).

Styrene Block Copolymer

The thermoplastic polymer can be a styrene block copolymer.

The styrene block copolymer has at least one A block that includes styrene and at least one B block that includes, e.g., elastomeric conjugated dienes (e.g., hydrogenated and unhydrogenated conjugated dienes), sesquiterpenes (e.g., hydrogenated and nonhydrogenated sesquiterpenes), and combinations thereof. The A blocks and the B blocks bind to one another in any manner of binding such that the resulting copolymer exhibits a variety of structures including, e.g., random, straight-chained, branched, radial, star, comb, tapered, and combinations thereof. The block copolymer can exhibit any form including, e.g., linear A-B block, linear A-B-A block, linear A-(B-A) n-B multi-block, A-(B-A)n-A multi block and radial (A-B)n-Y block where Y is a multivalent compound and n is an integer of at least 3, tetrablock copolymer, e.g., A-B-A-B, and pentablock copolymers having a structure of A-B-A-B-A. The adhesive composition can include blends of at least two different block copolymers.

Suitable styrene A blocks include, e.g., styrene, alpha-methylstyrene, o-methylstyrene, m-methylstyrene, p-methylstyrene, p-tert-butylstyrene, 2,4-dimethylstyrene, 2,4,6-trimethylstyrene, and combinations thereof.

Suitable block elastomeric conjugated diene B blocks include, e.g., butadiene (e.g., polybutadiene), isoprene (e.g., polyisoprene), 2,3-dimethyl-1,3-butadiene, 1,3-pentadiene, 1,3-hexadiene, styrene butadiene copolymer, and combinations thereof, and hydrogenated versions thereof including, e.g., ethylene, propylene, butylene and combinations thereof. Suitable B block sesquiterpenes include, e.g., beta farnesene.

Useful styrene block copolymers include, e.g., styrene-butadiene (SB), styrene-butadiene-styrene (SBS), styrene-isoprene block (SI), styrene-isoprene-styrene (SIS), styrene-ethylene-butylene-styrene (SEBS), styrene-ethylene-propylene-styrene (SEPS), styrene-isobutylene-styrene, styrene-butadiene-butylene-styrene (SBBS) and combinations thereof. Particularly useful block copolymers include styrene-butadiene-styrene, styrene-isoprene-styrene, and combinations thereof.

The styrene block copolymer can include more than one styrene block copolymer. When more than one styrene block copolymer is included the styrene content, the diblock content and the melt flow rate ranges specified below are a weight average of all the grades present.

As an example, if the hot melt adhesive composition comprises two styrene block copolymers A and B. Polymer A is present at 25 weight % (wA) with a styrene content of 15% (sA) and polymer B is present at 25 weight % (wB) with a styrene content of 20 weight % (sB). The average styrene content of the styrene block copolymer is calculated in the following way:

wA/(wA+wB)*sA+wB/(wA+wB)*sB=0.5(15)+0.5(20)=17.5 weight %.

The styrene block copolymer can have an average styrene content of from 20% by weight to 75% by weight, or even from 20% by weight to 45% by weight.

The styrene block copolymer can include from 0% by weight to 50%, 5% by weight to 50% by weight, or even from 10% by weight to 40% by weight diblock.

The styrene block copolymer can have an average Melt Flow Rate (MFR) per ASTM D 1238 (200° C./5 kg) in g/10 min of from 0.5 to 40, 4 to 35, or even 8 to 30. Useful block copolymers are commercially available under the VECTOR series of trade designations from Taiwan Synthetic Rubber Corporation (TSRC) (Taipei City, Taiwan) including VECTOR 4211 and DPX-660 styrene-isoprene-styrene block copolymers, under the KIBITON trade designation from Chi mei Corporation (Tainan City, Taiwan) including KIBITON PB-5502, under the GLOBALPRENE trade designation from LCY group (Taipei City, Taiwan) including GLOBALPRENE 3546, JH-8151 from Ningbo Jinhai Chenguang Chemical Corporation (China) and under the STYROFLEX trade designation from Ineos Styrolution (Frankfort, Germany) including STYROFLEX 2G66, S-TPE (styrene-butadiene with the properties of a thermoplastic elastomer).

The hot melt adhesive composition can include from 5% by weight to 50% by weight, from 10% by weight to 40% by weight, from 10% by weight to 30% by weight, from 10% by weight to 25% by weight, from 12% by weight to 25% by weight, or even from 15% by weight to 25% by weight, of a styrene block copolymer.

Bio-Based Tackifying Agent

The hot melt adhesive composition includes a bio-based tackifying agent. The bio-based tackifying agent can include one or more bio-based tackifying agents. The bio-based tackifying agent can be a liquid or a solid, however preference is given to bio-based tackifying agents that are solid at room temperature (18-26° C.). The bio-based tackifying agent can have a Ring and Ball Softening Point as reported by the supplier of at least 80° C., at least 90° C., from 80° C. to 140° C., from 80° C. to 120° C., or even from 80° C. to 105° C.

Useful bio-based tackifying agents can include terpene based tackifying agents (e.g., terpenes, modified terpenes and hydrogenated versions thereof) and rosin based tackifying agents (e.g. natural rosins, modified rosins, rosin esters, and hydrogenated versions thereof), sucrose benzoate, and oligomeric resins derived from other bio sources (e.g. isosorbide, isomannide, lignin, etc.).

The bio-based tackifying agents (e.g. terpene based tackifying agents, rosin based tackifying agents, etc.) can be modified with materials such styrene, phenol, carboxylic acids, anhydrides (e.g., maleic anhydride) and combinations thereof.

Examples of useful terpene and modified terpenes include those derived from alpha-pinene, beta-pinene, gamma-limonene, dipentene, or mixtures thereof.

Examples of useful rosin based tackifying agents include natural and modified rosins (e.g. disproportionated), including gum rosin, wood rosin, tall oil rosin, distilled rosin, hydrogenated rosin, dimerized rosin and polymerized rosin (e.g. rosin esters). Examples of useful rosin esters include e.g., glycerol esters of pale wood rosin, glycerol esters of hydrogenated rosin, glycerol esters of polymerized rosin, pentaerythritol esters of natural and modified rosins including pentaerythritol esters of pale wood rosin, pentaerythritol esters of hydrogenated rosin, pentaerythritol esters of tall oil rosin, and phenolic-modified pentaerythritol esters of rosin.

Useful rosin based tackifying agents include near water white rosin ester tackifying agents obtained by the processes taught in U.S. Ser. No. 10/611,926B2 and US2020199408A1 which are in hereby incorporated by reference.

Bio-based tackifying agents having a relatively low neat Molten Gardner Color are preferred. The bio-based tackifying agents can have neat Molten Gardner Color of no greater than 4, no greater than 2, no greater than 1, from 0 to 4, or even from 0 to 2.

In one embodiment, the bio-based tackifying agent is a rosin based tackifying agent having a neat Molten Gardner Color of no greater than 4, no greater than 2, no greater than 1, from 0 to 4, or even from 0 to 2

The hot melt adhesive composition can include from 10% by weight to 80% by weight, from 15% by weight to 75% by weight, from 20% by weight to 75% by weight, from 20% by weight to 70% by weight, from 35% by weight to 70% by weight, from 45% by weight to 70% by weight, or even from 50% by weight to 65% by weight of environmentally conscious, or even bio-based based tackifying agent.

Useful bio-based tackifying agents are commercially available under a variety of trade designations including rosin ester tackifying agents available under the SYLVALITE trade designation from Kraton Corporation (USA) such as e.g. SYLVALITE RE 100L, SYLVALITE 9100 and SYLVALITE RE 105L and under the KOMOTAC trade designation from Guangdong Komo Co. Ltd. such as e.g. KOMOTAC KM-100 and terpene tackifying agents available under the SYLVARES trade designation from Kraton Corporation (USA) such as e.g. SYLVARES 6100, SYLVARES TR M1115 and SYLVARES TP 2040 and the PICCOLYTE trade designation from DRT (France) such as PICCOLYTE S85 and PICCOLYTE F105IG

### Environmentally Conscious Aliphatic Plasticizer

The hot melt adhesive composition includes an environmentally conscious aliphatic plasticizer having a cyclo-aliphatic content of no more than 2% by weight, no more than 1% by weight, no more than 0.5% by weight, from 0% by weight to 2%, from 0% by weight to 1% by weight, or even from 0% by weight to 0.5% by weight as tested by 1H-NMR Spectroscopy. The environmentally conscious aliphatic plasticizer can have a CO2 emission value less than 1.0 kg CO₂ e/kg, or even less than 0 kgCO₂ e/kg.

In a preferred embodiment, the environmentally conscious aliphatic plasticizer is both bio-based and has a total cradle to gate CO₂ emission value of less than 1.5 kgCO₂ e/kg, less than 1.0 kgCO₂ e/kg, less than 0.5 kgCO₂e/kg, or even less than 0 kgCO₂ e/kg

The environmentally conscious aliphatic plasticizer can be present at from 2% by weight to 60% by weight, 5% by weight to 50% by weight, from 5% by weight to 40% by weight, from 5% by weight to 35% by weight, from 8% by weight to 35% by weight, from 8% by weight to 30% by weight, from 10% by weight to 30% by weight, or even from 12% by weight to 30% by weight.

Bio-Based Aliphatic Plasticizer

The environmentally conscious aliphatic plasticizer having a cyclo-aliphatic content of no more than 2% by weight as tested by ¹H-NMR Spectroscopy can be bio-based. The bio-based aliphatic plasticizer can be selected from the group consisted of linear alkanes, branched alkanes, or combinations thereof. The bio-based aliphatic plasticizer can be the hydrogenated reaction product of octadecane and hexadecane. The bio-based aliphatic plasticizer is low in cycloaliphatic and aromatic ring structures. The bio-based aliphatic plasticizer can include one or more bio-based aliphatic plasticizers.

The inventors have found that when used in combination with a bio-based tackifying agent, a composition including a high percentage of bio-based components and having performance over a broad temperature range as witnessed by a broader Temperature Plateau Range can be achieved.

The bio-based aliphatic plasticizer can have cyclo-aliphatic content of no greater than 2% by weight, no greater than 1.5% by weight, no greater than 1% by weight, no greater than 0.75% by weight, no greater than 0.5% by weight, no greater than 0.25% by weight, from 0% by weight to 2% by weight, from 0% by weight to 1% by weight, from 0% by weight to 0.5% by weight, or even from 0% by weight to 0.25% by weight as tested by 1H-NMR Spectroscopy.

The bio-based aliphatic plasticizer is derived from renewable resources e.g. bacteria, fermentation material, animal oil, plant oil (e.g. canola oil, corn oil, soybean oil, epoxidized soybean oil, palm oil, nut oil (e.g. peanut oil, cashew oil, etc.), olive oil, sunflower oil, rapeseed oil, jatropha oil, coconut oil, castor oil, etc.).

The bio-based aliphatic plasticizer can have a bio-based content of from 25% by weight to 100% by weight, from 50% by weight to 100% by weight, from 75% by weight to 100% by weight, or even 100% by weight bio-based content.

The bio-based aliphatic plasticizer can be present at from 2% by weight to 60% by weight, 5% by weight to 50% by weight, from 5% by weight to 40% by weight, from 5% by weight to 35% by weight, from 8% by weight to 35% by weight, from 8% by weight to 30% by weight, from 10% by weight to 30% by weight, or even from 12% by weight to 30% by weight.

Useful bio-based aliphatic plasticizer is available under the VIVASPES trade designation from H&R Group US, Inc. (Houston, Tex.)) including e.g., VIVASPES 10227 and VIVASPES 10229.

### Non-Environmentally Conscious Aliphatic Plasticizer

The hot melt adhesive composition can further include non-environmentally conscious aliphatic plasticizers. The non-environmentally conscious aliphatic plasticizer is low in cycloaliphatic and aromatic ring structures. The non-environmentally conscious aliphatic plasticizer can include one or more aliphatic plasticizers

Non-environmentally conscious aliphatic plasticizers can be selected from the group consisting of aliphatic oil, white mineral oil, paraffin oil, gas to liquid (GTL) oil, synthetic liquid oligomers of polyolefins (e.g. polyisobutylene, polybutene and polypropylene, etc.), hydrocarbon fluids, functionalized versions thereof, hydrogenated or hydro treated versions thereof and combinations thereof.

The non-environmentally conscious aliphatic plasticizer can have cyclo-aliphatic content of no more than 2% by weight, no more than 1% by weight, no more than 0.5% by weight, from 0% by weight to 2%, from 0% by weight to 1% by weight, or even from 0% by weight to 0.5% by weight as tested by ¹H-NMR Spectroscopy.

Additional non environmentally conscious aliphatic plasticizer can be present at from 2% by weight to 50% by weight, from 2% by weight to 30% by weight, from 2% by weight to 25% by weight, or even from 2% by weight to 20% by weight.

Useful additional aliphatic plasticizers are available under the DURASYN trade designation from INEOS Chemicals Co (London, UK) including e.g., DURASYN 166, white mineral oil under the PURETOL trade designation from Petro-Canada Lubricants Inc. (Mississauga, Ontario) including e.g., PURETOL 35 and TPC1160, a polyisobutylene available from TPC Group (Houston, Tex.)

### Wax

The hot melt adhesive composition can be free of a wax, alternatively the hot melt adhesive composition can include a wax. As with the polymer, the wax can be environmentally conscious or even bio-based.

Useful classes of wax include, e.g., paraffin waxes, microcrystalline waxes, high density low molecular weight polyethylene waxes, by-product polyethylene waxes, polypropylene waxes, Fischer-Tropsch waxes, oxidized Fischer-Tropsch waxes, functionalized waxes such as acid, anhydride, and hydroxyl modified waxes, animal waxes, vegetable waxes (e.g. soy wax) and combinations thereof.

Useful waxes are solid at room temperature and preferably have a Ring and Ball softening point of from 50° C. to 170° C.

The wax can be a propylene based wax with a Mettler Softening Point of greater than 130° C., greater than 140° C., or even greater than 150° C. Useful waxes are commercially available from a variety of suppliers including polypropylene and polyethylene waxes available under the EPOLENE N and C series of trade designations from Westlake Chemical Corporation (Houston, Tex.) including e.g. EPOLENE N-21, EPOLENE N-15 and polypropylene and polyethylene waxes available under the LICOCENE series of trade designations from Clariant International Ltd. (Muttenz, Switzerland) including e.g. LICOCENE PP 6102, LICOCENE PP 6502 TP and LICOCENE PP 7502 TP and Fischer-Tropsch wax available under the SARAWAX series of trade designations from Shell MDS (Malaysia) Sdn Bhd including GTL SARAWAX SX105.

The hot melt adhesive composition can include no greater than 10% by weight, no greater than 5% by weight, from 2% by weight to 10% by weight, or even from 3% to 8% by weight wax.

### Additional Tackifying Agents

The hot melt adhesive composition can include additional tackifying agents including those derived from petroleum-based feedstocks. Examples of useful additional tackifying agents include hydrocarbon tackifying agents. Hydrocarbon tackifying agents include, e.g., aromatic, aliphatic and cycloaliphatic hydrocarbon resins, and hydrogenated versions thereof, aromatic modified aliphatic or cycloaliphatic hydrocarbon resins, and hydrogenated versions thereof; and combinations thereof.

Examples of useful aliphatic and cycloaliphatic petroleum hydrocarbon resins include aliphatic and cycloaliphatic petroleum hydrocarbon resins include, e.g., branched and unbranched C9 resins and C10 resins and the hydrogenated derivatives thereof.

Additional tackifying agents are present at no more than 20% by weight, no more than 15% by weight, no more than 10% by weight, no more than 5% by weight, from 0% by weight to 20% by weight, or even from 5% by weight to 20% by weight.

### Additional Components

The hot adhesive composition optionally includes additional components including, e.g., petroleum derived tackifying agents, additional polymers (e.g. olefin polymers, ethylene vinyl copolymers (e.g. ethylene vinyl acetate)), limited amounts of plasticizers having a cycloaliphatic content of greater than 2% by weight as tested by ¹H-NMR Spectroscopy e.g. naphthenic oil, standard vegetable oils, etc., stabilizers, antioxidants, adhesion promoters, ultraviolet light stabilizers, colorants (e.g., pigments and dyes), fillers, surfactants, perfumes, lotions, co-extrusion coatings, packaging films, wetness indicators, superabsorbents and combinations thereof.

Useful antioxidants include, e.g., pentaerythritol tetrakis[3,(3,5-di-tert-butyl-4-hydroxyphenyl)propionate], 2,2′-methylene bis(4-methyl-6-tert-butylphenol), phosphites including, e.g., tris-(p-nonylphenyl)-phosphite (TNPP) and bis(2,4-di-tert-butylphenyl)4,4′-diphenylene-diphosphonite, di-stearyl-3,3′-thiodipropionate (DSTDP), and combinations thereof. Useful antioxidants are commercially available under a variety of trade designations including, e.g., the IRGANOX series of trade designations including, e.g., IRGANOX 1010, IRGANOX 565, and IRGANOX 1076 hindered phenolic antioxidants and IRGAFOS 168 phosphite antioxidant, all of which are available from BASF Corporation (Florham Park, N.J.), and Ethyl 702 4,4′-methylene bis(2,6-di-tert-butylphenol). When present, the adhesive composition preferably includes from 0.1% by weight to 3% by weight antioxidant.

### USES

The hot melt adhesive compositions of this invention can be used in many different applications and for a variety of end uses including pressure sensitive adhesives (e.g. removable and permanent types), bookbinding adhesives, adhesives to attach inserts to published materials (e.g. magazines), adhesives to assemble various items (e.g. filters), adhesives for packaging constructions (e.g. cases, cartons, trays, etc.), adhesives for tapes and labels, and adhesives for disposable articles.

### Tapes and Labels

The hot melt adhesive compositions of this invention can be used to make adhesive tapes or alternately to adhere labels to various items (e.g. containers, magazines, etc.). The label/tape can be selected from a variety of materials including paper, non-paper films (e.g. polypropylene (e.g. polypropylene (PP), oriented polypropylene (OP), and biaxially oriented polypropylene (BOPP)), polyethylene, etc.). The container can be metal (e.g. aluminum or steel) or plastic (polyethylene terephthalate (PET), high density polyethylene (HDPE) and polypropylene.

The label can be a spot label i.e. a label that does not extend completely around the container. Alternatively, the label can be a wraparound label i.e. a label that completely wraps around the entire container.

If the label is a wraparound label, it can be roll fed into the applicator. Alternatively, the labels are pre-cut and fed in from a stack. In a wraparound label application method, the label stock is fed into a label station. A pick-up adhesive and a lap glue are then applied to the label, often from the same glue pot. A pick-up adhesive adheres the leading edge of the label to a container. The lap glue then bonds the overlap where the wrap around label overlaps itself. The hot melt adhesive composition of this invention can be both the pick-up adhesive and the lap glue.

### Disposable Absorbent Articles

The hot melt adhesive composition can be applied to (i.e. such that it is in direct contact with) or incorporated in a variety of substrates including, e.g., films (e.g., polyolefin (e.g., polyethylene and polypropylene), bio-based films), release liners, porous substrates, cellulose substrates, sheets (e.g., paper, and fiber sheets), paper products, woven and nonwoven webs, fibers (e.g., synthetic polymer fibers and cellulose fibers) and tape backings.

The hot melt adhesive composition is also useful in a variety of applications and constructions including, e.g., disposable absorbent articles including, e.g., disposable diapers, adult incontinence products, sanitary napkins, medical dressings (e.g., wound care products) bandages, surgical pads, pet training pads (e.g. puppy pads) and meat-packing products and components of absorbent articles including, e.g., an absorbent element, absorbent cores, impermeable layers (e.g., backsheets), tissue (e.g., wrapping tissue), acquisition layers and woven and nonwoven web layers (e.g., top sheets, absorbent tissue) and elastics.

The hot melt adhesive composition is useful on substrates made from a variety of fibers including, e.g., natural cellulose fibers (e.g. wood pulp, cotton, viscose, starch, etc.), silk, PLA(poly lactic acid), PHA(poly hydroxyl alkanoates), PBS(poly butylene succinate), PBAT (poly butylene adipate terephthalate) and wool; synthetic fibers such as nylon, rayon, polyesters, acrylics, polypropylenes, polyethylene, polyvinyl chloride, polyurethane, and glass; recycled fibers, and various combinations thereof.

The hot melt adhesive composition is useful on a variety of films including polyethylene, polypropylene, ethylene vinyl acetate, ethylene copolymer, bio-based films (e.g. PLA, PHA, starch, etc.).

Various application techniques can be used to apply the composition to a substrate including, e.g., slot coating, spraying including, e.g., spiral spraying and random spraying, screen printing, foaming, engraved roller, extrusion and melt blown application techniques.

### Methods of Making Disposable Absorbent Articles

The hot melt adhesive compositions of this invention can be used in a wide variety of applications within the disposable absorbent article. The hot melt adhesive compositions can be used as construction adhesives (e.g. used to bond the back sheet to the nonwoven and optionally the absorbent pad), as a positioning adhesives (e.g. to adhere a disposable absorbent article to an undergarment), as an elastic attachment adhesive (e.g. bonding the elastic material to the back sheet in for example the leg or waist area), or to attach elastic material to any other portion of the article, and for core stabilization (e.g. applying a hot melt composition to the absorbent core to increase the strength of the core).

The hot melt adhesive composition can be used for construction applications. In a typical construction application in the manufacture of a disposable absorbent article, a body fluid impermeable back sheet is bonded to a nonwoven substrate. The hot melt adhesive composition may also be used to bond at least one additional layer or material selected from the group consisting of absorbents, tissues, elastomeric materials, superabsorbent polymers, and combinations thereof. For example, the adhesive can further be used for back sheet lamination i.e. where the body fluid impermeable backsheet typically a film (e.g. polyethylene, polypropylene, ethylene vinyl acetate, ethylene copolymer, bio-based etc.) is bonded to a second nonwoven to improve the feel of the disposable article.

The hot melt adhesive composition can be used as a positioning adhesive. A positioning adhesive is disposed on at least one substrate surface of a disposable absorbent article and can be used to position an absorbent article on a garment such as underwear. Such disposable absorbent articles include, e.g., feminine hygiene articles such as sanitary napkins and panty liners, diapers, disposable garments having a waist opening and leg openings, and adult incontinence articles. In one construction, the absorbent article (e.g., a feminine hygiene article) includes a garment facing surface and a body facing surface, a topsheet having a garment facing surface and a body facing surface, a backsheet having a garment facing surface and a body facing surface, and an absorbent core disposed between the body facing surface of the backsheet and the garment facing surface of the topsheet. The pressure-sensitive adhesive composition is disposed on the garment facing surface of the adsorbent article, in general, or even on the garment facing surface of the backsheet. A release liner optionally is disposed on the pressure-sensitive hot melt adhesive composition to protect the pressure-sensitive adhesive composition until use. The absorbent article (e.g., a feminine hygiene article) optionally includes additional layers and adhesives and the components of the absorbent article optionally exhibit additional functionality. Examples of additional layers, functionality and combinations thereof include dusting, wicking, acquisition, additional top sheets, multiple core layers, superabsorbent particles and compositions, wetness indicators, and combinations thereof.

The invention will now be described by way of the following examples. All parts, ratios, percents and amounts stated in the Examples are by weight unless otherwise specified.

### EXAMPLES

Test procedures used in the examples and throughout the specification, unless stated otherwise, include the following.

### Brookfield Viscosity Test Method

Viscosity was determined in accordance with ASTM D-3236 entitled, “Standard Test Method for Apparent viscosity of Adhesives and Coating Materials,” (Oct. 31, 1988), using a Brookfield Thermoset viscometer Model DV1 2 and a number 27 spindle. The results were reported in centipoise (cP).

### Glass Transition Temperature (Tg) Test Method

The glass transition temperature (Tg) of the samples was determined using Dynamic Mechanical Analysis (DMA) with a DHR-II instrument using the following conditions: the sample was heated to 150° C., held at 150° C. for 5 minute, cooled to −20° C. at 3° C./minute and 10 rad/second, with 10% strain. The Tg was the temperature at which the tan delta curve exhibits a local maxima at material transition zone between the glassy and rubbery regions, usually between −20° C. and 40° C.

### Temperature Plateau Range

The temperature plateau range was obtained using Dynamic Mechanical Analysis (DMA) with a DHR-II instrument using the following conditions: the sample was heated to 150° C., held at 150° C. for 5 minute, cooled to −20° C. at 3° C./minute and 10 rad/second, with 10% strain.

As temperature decreases from 150° C., storage modulus G′ and loss modulus G″ increase. The temperature, at which both these two curves cross over, was called first crossover-temperature (T1). This was where material transitions from melt to rubbery plateau region. As temperature continues to decrease, G′ and G″ curves cross over again, when material starts to transition into glassy state. This temperature was called second crossover-temperature (T2). T1 and T2 define the temperature plateau range.

### Cycloaliphatic Content

Cycloaliphatic content was obtained using H-1 Nuclear Magnetic Resonance (1H-NMR) Spectroscopy. The summation of the signals from 1.9-2.5 ppm was calculated as a percentage from the total aliphatic hydrogen summation from 0.2-3.7 ppm. If antioxidant or other additives were present in the oil sample, the respective resonances for these additives were omitted from the integration regions defined.

### Molten Gardner Color

The adhesive is tested (in the molten state) to determine Gardner color by comparing the color of the sample against the Gardner Color Standards as set forth in ASTM D-1544. The comparison is made using a Gardner Delta Comparator equipped with an Illuminator available from Pacific Scientific (Bethesda, Md.).

### Dynamic Peel Test Sample Preparation Method

A slot coating applicator, which was 1 inch (25.4 mm) wide, and a laminator were set to an application temperature of 149° C., a nip pressure of 103.4 kilopascal (15 psi), and minimal rewind and unwind tensions so as not to stretch the film. The hot melt adhesive composition was applied continuously at a coat weight of 3 g/m2 on an embossed non-breathable, layered polyethylene film having a thickness of 0.9 mil (0.023 mm), and laminated with an oriented polypropylene nonwoven web having a thickness of 4 mil (0.1 mm) and a basis weight of 0.45 ounces per square yard (15.3 g/m2) at a speed of around 184 m/min.

### Dynamic Peel Test Method

Dynamic Peel was determined according to ASTM D1876-01 entitled, “Test Method for Determining Peel Resistance of Adhesive (T-Peel Test Method),” with the exception that the test was run at 30.5 centimeters per minute (12 in/min) over a period of 10 seconds and 6 replicates were run. The samples were run on an IMASS Spec-type test instrument. The samples were peeled along the machine coating direction. The average peel value over 10 seconds of peeling was recorded, and the results were reported in grams. The initial Dynamic Peel value was the value measured 24 hours after the sample was prepared. Six replicates were tested and the average value was reported in units of grams of force per 25 mm (gf/25 mm).

### Sample Preparation Method for Cotton Peel Force Test

A laminate was prepared by coating a sample composition onto a silicone coated release paper in a one inch wide pattern at an add-on weight of 20 grams per square meter (g/m2) (+/−3 g/m2) using a slot applicator and then contacting the adhesive strip with the treated side of a 0.9 mil (0.023 mm) thick polyethylene film to form a silicone coated release paper/adhesive/polyethylene film laminate. Test samples having a length of 4 inches (in) (10.16 cm) in the machine coating direction and 2 in (5.08 cm) in the cross-machine direction were then cut from the laminate such that the adhesive pattern was centered in the cross-machine direction of the test sample.

### Cotton Peel Force Test Method

For cotton bonds a sheet of 124 g/m2 bleached t-shirt cotton fabric (Testfabrics, Inc., West Pittston, Pa.) was cut into strips having a length of 4 in (10.16 cm) in the machine direction and a width of 1.5 in (3.81 cm) in the cross-machine, before cutting the cotton fabric, the grid work of the stitching of the fabric was examined. When the cotton fabric was stretched, the sample will exhibit greater elongation in one direction than in another direction. The cotton fabric was cut lengthwise in the direction that has less elongation. All cotton fabric strips were cut as straight as possible along the stitching grid work. If the cotton fabric strips were cut askew, an inconsistent elongation of the cotton fabric test sample will result.

The release film was removed from the adhesive and the adhesive side of each test sample was gently placed on the surface of a cotton strip such that the cotton curls away from the adhesive bond to form the composite test sample, taking care not to press the adhesive down on to the test fabric. That was, the easier to bond to side of the cotton fabrics was used in peel testing.

The resulting test specimen was then placed on a mechanical roll-down device equipped with a 4.5-pound roller, and the roller was allowed to pass two times over the sample, i.e., one forward pass and one backward pass, at a rate of approximately 12 in/min (305 mm/min) once in each lengthwise direction, ensuring that the sample is free of entrapped air bubbles.

A timer was then activated, and the sample was placed into the jaws of INSTRON-type peel tester. The polyethylene film was placed into the moving jaw, and the fabric was attached to the stationary jaw. Within one minute after the sample has been removed from the roll-down device, the sample was tested according to ASTM D1876-01 entitled, “Test Method for Determining Peel Resistance of Adhesive (T-Peel Test Method),” with the exception that the test is run at a rate of 305 mm/min, instead of 250 mm/min, over a period of ten seconds, and at least five replicates were run instead of the ten specified in ASTM D1876. The average peel force over ten seconds of peeling was recorded, and the results were reported in grams.

The initial peel force was measured 24 hours after the test sample was prepared.

The following grades of styrene block copolymer are used in the Examples.

JH8151—SIS, 16% by weight styrene, MFR=10 (200° C., 5 kg)

KIBITON PB 5502—SBS, 36.5% by weight styrene, MFR=8 (190° C., 2.16 kg)

VECTOR 4211—SIS, 30% by weight styrene, MFR=13 (200° C., 2.16 kg)

GLOBALPRENE 3546—SBS, 40% styrene, MFR=6 (200° C., 2.16 kg)

STYROFLEX 2G66—S-TPE-S, 64% styrene, MFR=11 (200° C., 5 kg)

TABLE ONE

| | Control 1 | Ex 1 |
|---|---|---|
| JH8151 | 8 | 8 |
| KIBITON PB 5502 | 12 | 12 |
| Average SBC Styrene | 28.3 | 28.3 |
| content (% by weight) | | |
| SYLVALITE 9100 | 60 | 60 |
| Antioxidant | 1 | 1 |
| Co-extrusion coating | 0.5 | 0.5 |
| Packaging film | 0.5 | 0.5 |
| AGRIPURE 60 | 18 | |
| VIVASPES 10227 | | 18 |
| Cycloaliphatic content of | Not applicable | 0 |
| plasticizer (weight %) | Note: | |
| | AGRIPURE 60 is 60% | |
| | oleic acid and is | |
| | derived from canola oil | |
| Brookfield Viscosity @ | 3250 | 3650 |
| 149° C. (cP) | | |
| Temperature Plateau Range (° C.) | 20-51 (31) | 22-76 (54) |
| Environmentally conscious | 78 | 78 |
| component (wt. %) | | |
| Bio-based component (wt. %) | 78 | 78 |

TABLE TWO

| | Control 2 | Ex 2 | Control 3 | Ex 3 | Control 4 | Ex 4 | Ex 5 |
|---|---|---|---|---|---|---|---|
| JH8151 | 8 | 8 | 8 | 8 | 8 | 8 | 8 |
| KIBITON PB 5502 | 12 | 12 | 12 | 12 | 12 | 12 | 12 |
| Average SBC Styrene | 27.3 | 27.3 | 27.3 | 27.3 | 27.3 | 27.3 | 27.3 |
| content (% by weight) | | | | | | | |
| SYLVARES 6100 | 60 | 60 | | | | | 60 |
| SYLVARES TR M1115 | | | 60 | 60 | | | |
| SYLVARES TP 2040 | | | | | 60 | 60 | |
| Antioxidant | 1 | 1 | 1 | 1 | 1 | 1 | 1 |
| Co-extrusion coating | 0.5 | 0.5 | 0.5 | 0.5 | 0.5 | 0.5 | 0.5 |
| Packaging film | 0.5 | 0.5 | 0.5 | 0.5 | 0.5 | 0.5 | 0.5 |
| CALSOL 5550 | 18 | | 18 | | 18 | | |
| VIVASPES 10227 | | 18 | | 18 | | 18 | |
| VIVASPES 10229 | | | | | | | 18 |
| Cycloaliphatic content | 3.5 | 0 | 3.5 | 0 | 3.5 | 0 | 0.1 |
| of plasticizer (%) | | | | | | | |
| Brookfield Viscosity @ | 3325 | 3250 | 9850 | 13950 | 4575 | 4125 | 2675 |
| 149° C. (cP) | | | | | | | |
| Temperature Plateau | 42-72 | 28-83 | 42-72 | 51-114 | <−20*to75 | <−20*to89 | 15-67 |
| Range (° C.) | (30) | (55) | (30) | (63) | (95) | (119) | (52) |
| Environmentally conscious | 60 | 78 | 60 | 78 | 60 | 78 | 78 |
| component (wt. %) | | | | | | | |
| Bio-based component (wt. %) | 60 | 78 | 60 | 78 | 60 | 78 | 78 |

*beyond instrument low temperature limit

TABLE THREE

| | Ex 6 | Ex 7 | Ex 8 | Ex 10 |
|---|---|---|---|---|
| JH8151 | 10 | 8 | 10 | |
| KIBITON PB5502 | 12 | 12 | | |
| VECTOR 4211 | | | | 6 |
| GLOBALPRENE 3546 | | | | 19 |
| STYROFLEX 2G66 | | | 10 | |
| Avg Styrene content | 27.3 | 28.3 | 40 | 37.6 |
| SYLVALITE RE 100L | | | | 61 |
| SYLVALITE 9100 | 55 | 60 | 59.5 | |
| Antioxidant | 1 | 1 | 1 | 1 |
| Co-extrusion coating | 0.5 | 0.5 | 0.5 | 0.5 |
| Packaging film | 0.5 | 0.5 | 0.5 | 0.5 |
| GTL SARAWAX SX105 | | | 0.5 | |
| VIVASPES 10227 | 21 | 18 | 18 | 12 |
| Brookfield Viscosity @ | 4250 | 3650 | 7100 | 10150 |
| 149° C. (cP) | | | | |
| Temperature Plateau | 8-81 (73) | 22-76 (54) | 22-63 (41) | 24-83 (59) |
| Range (° C.) | | | | |
| Environmentally | 76 | 78 | 77.5 | 73 |
| conscious | | | | |
| component (wt. %) | | | | |
| Bio-based | 76 | 78 | 77.5 | 73 |
| component (wt. %) | | | | |
| Glass Transition | −15 | −5 | −1 | 8 |
| Temperature (° C.) | | | | |
| Peel Adhesion to | 322 | 451 | 283 | NT |
| Cotton (g/in) | | | | |
| Dynamic Peel | NT | NT | NT | 116 |
| Adhesion (g/in) | | | | |

NT (not tested)

The adhesive compositions were prepared by combining and mixing the components in the percentages set forth in Table 1, 2, and 3 in a sigma blade mixer operating at 177° C.

What is claimed is:

1. A hot melt adhesive composition comprising:
a. from 5% by weight to 50% by weight of a thermoplastic polymer,
b. from 10% by weight to 80% by weight of a bio-based tackifying agent, and
c. from 2% by weight to 50% by weight of an environmentally conscious aliphatic plasticizer having a cyclo-aliphatic content of no more than 2% by weight as tested by ¹H-NMR Spectroscopy.

2. The hot melt adhesive composition of claim 1 wherein the environmentally conscious aliphatic plasticizer is a bio-based aliphatic plasticizer.

3. The hot melt adhesive composition of claim 2 wherein the thermoplastic polymer is selected from the group consisting of olefin polymer, styrene block copolymer, functionalized versions thereof and combinations thereof.

4. The hot melt adhesive composition of claim 1 having an environmentally conscious component content of from 65% by weight to 100% by weight.

5. The hot melt adhesive composition of claim 2 having a bio-based component content of from 65% by weight to 100% by weight.

6. The hot melt adhesive composition of claim 2 wherein the bio-based aliphatic plasticizer is derived from plant oil.

7. The hot melt adhesive composition of claim 2 wherein the bio-based aliphatic plasticizer is a hydrogenated reaction product of octadecane and hexadecane.

8. The hot melt adhesive composition of claim 2 wherein 100% by weight of the bio-based aliphatic plasticizer is produced or derived from renewable resources.

9. The hot melt adhesive composition of claim 2 wherein the bio-based aliphatic plasticizer has a cyclo-aliphatic content of no more than 1% by weight as tested by ¹H-NMR Spectroscopy.

10. The hot melt adhesive composition of claim 2 wherein the bio-based aliphatic plasticizer has a cyclo-aliphatic content of no more than 0.5% by weight as tested by ¹H-NMR Spectroscopy.

11. The hot melt adhesive composition of claim 1 wherein the bio-based tackifying agent is selected from the group consisting of rosin-based tackifying agent and terpene-based tackifying agent.

12. The hot melt adhesive composition of claim 1 wherein the bio-based tackifying agent is a rosin-based tackifying agent.

13. The hot melt adhesive composition of claim 1 wherein 70% by weight to 100% by weight of the bio-based tackifying agent is produced or derived from renewable resources.

14. The hot melt adhesive composition of claim 1 wherein the thermoplastic polymer is selected from the group consisting of bio-based and thermoplastic polymers considered sustainable by the mass balance approach

15. The hot melt adhesive composition of claim 1 wherein the thermoplastic polymer is a styrene block copolymer.

16. The hot melt adhesive composition of claim 2 comprising:
a. from 10% by weight to 40% by weight of the thermoplastic polymer, the thermoplastic polymer comprising styrene block copolymer,
b. from 15% by weight to 75% by weight of the bio-based tackifying agent, and
c. from 5% by weight to 40% by weight of the bio-based aliphatic plasticizer.

17. An article selected from the group consisting of a tape, a label and a disposable absorbent article comprising the hot melt adhesive composition of claim 1.

18. A disposable absorbent article comprising:
a.) a first substrate,
b.) a second substrate, and
c.) the hot melt adhesive composition of claim 1,
wherein the hot melt adhesive composition is disposed on at least one of the first and second substrates.

19. The disposable absorbent article of claim 18 wherein at least one of the substrates is bio-based.

20. The disposable absorbent article of claim 18 wherein the bio-based substrate is cotton.
